(19) United States
(12) Patent Application Publication
(10) Pub. No.: US 2005/0202090 A1
(43) Pub. Date: Sep. 15, 2005

(54) Novel pharmaceutical dosage forms and method for producing same

(76) Inventor: Allan J. Clarke, Collegeville, PA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/500,630

(22) PCT Filed: Jan. 3, 2003
(86) PCT No.: PCT/US03/00099

§ 371 Date: Jun. 29, 2004

(30) Foreign Application Priority Data
Jan. 3, 2002 (US) 60345526

(51) Int. Cl. A61K 9/14

(52) U.S. Cl. 424/486; 424/488

(57) ABSTRACT

Pharmaceutical dosage forms are produced by injection molding a mixture of an agent and a polymer under pressure, in the presence of a gas or supercritical fluid. Rapid release of the pressure causes the mixture to form a microcellular or supermicrocellular solid. The release of pressure takes place in the mold. The process is especially useful for producing durable flash-dissolve and gastro-retentive tablets.

### FIELD OF THE INVENTION

This invention relates generally to pharmaceutical dosage forms and their manufacture, and more particularly to a novel dosage form in which an active agent is combined with a solid excipient having a foamed structure.

### BACKGROUND OF THE INVENTION

Pharmaceutical preparations, especially solid preparations intended for oral administration, are frequently supplied in so-called “flash-dissolve” tablets, which dissolve almost immediately, i.e., within seconds, upon contact with saliva in the patient's mouth. Flash-dissolve tablets are particularly desirable for use as solid pediatric oral preparations and for administration to adult patients who have difficulty in swallowing tablets.

Flash-dissolve tablets typically utilize special, highly soluble formulations and disintegration promoters, and also have a high surface area-to-volume ratio to promote quick solution. In the past, because of their high friability, flash-dissolve tablets could not be subjected to post-formation handling, and to processing steps such as coating, ink-jet printing, etc., without breaking up. Therefore, it has been conventional practice to produce flash-dissolve tablets by freeze-drying the tablet material in the blisters of a blister package in which they were ultimately to be sold. The tablets took their shape from the blisters, and consequently the shape of the tablets was difficult to control.

In the case of a swallowed tablet, low density is desirable in order to make the tablet “gastro-retentive”. Unlike a heavier tablet, which would pass quickly into the duodenum, a low density tablet can float in the stomach while it dissolves slowly. A low-density, gastro-retentive tablet may be formed, for example, by pressing together grains of porous material formed by extruding a polymer containing a blowing agent and a drug substance, as described in European Patent Application 94924386.9, published on Jun. 26, 1996 under number EP 0 717 988 A1. Another gastro-retentive tablet is described in U.S. Pat. No. 6,312,726, granted on Nov. 6, 2001. In accordance with U.S. Pat. No. 6,312,726, an auxiliary blowing agent such as aluminum hydroxide gel, synthetic aluminosilicate, calcium hydrogen phosphate, calcium carbonate, sodium hydrogen carbonate, calcium hydrogen carbonate or talc, is used as an additive in order to generate a multiplicity of microfine pores or air spaces uniformly distributed within an extruded pharmaceutical product. The pores are described as having a mean diameter as small as 10-20 microns. Conventional low density, gastro-retentive tablets, however, have been prone to-weakness and tend to break apart in handling. Accordingly, they have been subject to problems similar to those encountered in the case of flash-dissolve tablets.

Various other porous tablets have been proposed. For example, U.S. Pat. No. 3,885,026, granted on May 20, 1975, describes tablets in which pores are formed by sublimation of an adjuvant such as urethane, urea, ammonium carbonate, etc. in a tablet formed in a tablet press. These tablets are porous, but the pores are in the form of comparatively large hollow spaces and canals through which a solvent can penetrate. They are readily dissolved, but are neither flash-dissolving nor gastro-retentive.

U.S. Pat. No. 6,150,424, granted Nov. 21, 2000, describes a process for extruding solid foamed thermoplastic polymer drug carriers with an active substance produced by melt-extrusion of an active ingredient such as ibuprofen in the thermoplastic binder, homo- or co-polymers of N-vinylpyrrolidone along with a blowing agent such as carbon dioxide, nitrogen, air, helium, argon, CFC or N₂O. This process introduces volatile blowing agents into the extrudate melt. The expanded extrudate is shaped into a dosage form after extrusion.

Another problem encountered in tablet manufacture is that tablets, including porous tablets of the kind described in European Patent Application 94924386.9, and U.S. Pat. No. 3,885,026, are formed by tablet presses. Such presses, although rapid in their operation, are very expensive. Furthermore, they must be shut down frequently for maintenance.

Attempts have been made to produce pharmaceutical tablets by injection molding, which was a promising alternative to the tablet press method. However, despite these attempts, injection molding has never been successful, and most tablets are still produced by tablet presses.

Various articles of manufacture, such as automobile dashboards, etc. have been formed from resins, such as PET, polystyrene, polyethylene, and PVC, which are expanded by a blowing agent, typically a low molecular weight organic compound mixed into a polymer matrix and heated to cause decomposition of the compound, resulting in the release of a gas (or gases) such as nitrogen, carbon dioxide, and carbon monoxide. Resins can also be expanded by physical processes not involving decomposition or other chemical reaction. For example, a gas may be introduced as a component of a polymer charge or introduced under pressure into a molten polymer.

These standard resin expansion processes produce foamed resins having cells which are relatively large, i.e., on the order of 100 microns, or larger, with the void fraction, that is the volume of the cells divided by the total volume, typically ranging from 20%-40% in structural foams, and from 80%-90% in insulation foams. The number of cells produced per unit volume is relatively low (on the order of 106 cells/cm³), and the size distribution of the cells is typically broad; that is the cell size is far from uniform throughout the foamed material.

A great deal of research and development work has been carried out on microcellular and supermicrocellular foam process technology. This technology has made it possible to produce expanded plastics having much smaller cells, and a much more narrow cell size distribution, with the result that the plastics exhibit a strength to weight ratio substantially greater than that of conventional foamed plastics. Microcellular foaming has proven useful in producing stable, small cell, materials at low cost, and products made from microcellular foams have been produced on a commercial scale.

Microcellular plastics are generally defined as foamed plastics characterized by cell sizes less than about 100 microns. Typical cell sizes are in the range from about 1 to 100 microns. Cell densities are typically on the order of 10⁹ cells per cubic centimeter. The specific densities are typically in the range from 5 to 95 percent of the density of the polymer, and the void fraction is similarly in the range of about 5 to 95 percent. These cells are smaller than the flaws preexisting within the polymers and, thus, do not compromise the polymers' specific mechanical properties. The result is a lower density material with no decrease in specific strength and a significant increase in toughness compared to the original polymers.

With a further reduction in cell size and an increase in cell density, supermicrocellular plastics can be produced, having cell sizes less than 1 micron, typically in the range from about 0.1 to 1.0 micron. Supermicrocellular plastics can have and cell densities greater than 10⁹ cells per cubic centimeter, and may be in the range of 10¹² to 10¹⁵ cells per cubic centimeter.

Either microcellular or supermicrocellular plastics may be used in the invention for producing solid oral dosage forms containing an active agent. Unless otherwise indicated, the term “microcellular,” as used herein, should be understood to encompass both microcellular and supermicrocellular materials.

Microcellular foams, and processes and equipment for making microcellular foams, are described in the following United States Patents:

| | | |
|---|---|---|
| 4,473,665 | Sep. 25, 1984 | Martini-Vvedensky et al. |
| 4,922,082 | May 1, 1990 | Bredt et al. |
| 5,158,986 | Oct. 27, 1992 | Cha et al. |
| 5,160,674 | Nov. 3, 1992 | Colton et al. |
| 5,334,356 | Aug. 2, 1994 | Baldwin et al. |
| 5,866,053 | Feb. 2, 1999 | Park et al. |
| 6,005,013 | Dec. 21, 1999 | Suh et al. |
| 6,051,174 | Apr. 18, 2000 | Park et al. |
| 6,231,942 | May 15, 2001 | Blizard et al. |
| 6,322,347 | Nov. 27, 2001 | Xu, J. |

and in published International patent applications WO 98/08667 and WO 99/32544. The disclosures of all of the above-listed patents and publications are here incorporated by reference in their entirety.

In general, microcellular foams are produced by injecting a gas, or a supercritical fluid (SCF), into a polymer while the polymer is under pressure and at an elevated temperature, and then reducing the pressure and temperature to cause a large number of cells to form in the polymer, and controlling the growth of the cells by appropriate processing conditions.

The production of microcellular foams is typically carried out by injecting a supercritical fluid, for example carbon dioxide, into a polymer while the polymer is maintained under an elevated pressure. A supercritical fluid is defined as a material maintained at a temperature exceeding a critical temperature and at a pressure exceeding a critical pressure so that the material is in a fluid state in which it exhibits properties of both a gas and a liquid. The supercritical fluid and the polymer form a single-phase solution. The pressure acting on the solution is then rapidly reduced, resulting in controlled nucleation at a very large number of nucleation sites. The gas then forms bubbles, the growth of which is controlled by carefully controlling pressure and temperature. The foams can be injection molded in conventional molding equipment.

Microcellular foam technology, although highly effective and useful for producing traditional articles of manufacture, such as automobile dashboards, etc., has not been applied to the pharmaceutical industry for injection molding of tablets. Apparently, the failures experienced by pharmaceutical manufacturers in attempts to produce tablets by injection molding have deterred them from going forward with research and development in the use of microcellular foam technology.

### BRIEF SUMMARY OF THE INVENTION

It has been determined that microcellular foam technology can in fact be utilized successfully in the production of pharmaceutical tablets, and that microcellular foam technology affords significant advantages, both in the manufacturing process and in the product itself. More particularly, microcellular foam can produce molded tablets having desirable properties and consistent quality, rapidly and at low cost.

In accordance with the invention, pharmaceutically acceptable dosage forms are made by the following steps. First, a non-thermosetting excipient polymer is supplied. The polymer is preferably pre-mixed with a pharmaceutical agent to form a homogeneous mixture, and heated to form an extrudable mass using a conventional, twin-screw extruder. To form the pharmaceutical dosage forms, the extruded polymer/pharmaceutical agent mixture is cut into pellets, which have a free-flowing property. The pellets are fed into the hopper of an injection molding machine, in which, while maintaining the polymer at elevated pressure, a single phase solution is formed, preferably by injecting into the polymer a substance which is a gas under ambient temperature and pressure, and which is substantially non-reactive with the pharmaceutical agent. The polymer, which has by this time been mixed homogeneously with the pharmaceutical agent, is then molded into solid dosage forms, and in the process of molding the solid dosage forms, the elevated pressure is reduced to a level at which cells are nucleated in large numbers, each cell containing the gas. After the cells are nucleated, the temperature of the polymer is rapidly reduced to limit cell growth.

The substance which is introduced into the polymer may be introduced in the form of a gas. The gas is preferably soluble in the polymer, and, where the gas is soluble, the level to which the elevated pressure is reduced must be a level at which the solution becomes thermodynamically unstable and the gas evolves from the solution in the form of bubbles. Alternatively, a gas which is not soluble in the polymer may be used, nitrogen being a typical example. The use of nitrogen is described in U.S. Pat. No. 5,034,171, whose disclosure is incorporated by reference in its entirety herein. In accordance with a preferred method, however, the substance introduced into the polymer is introduced, in the form of a supercritical fluid.

The pressure and temperature reduction steps are preferably carried out at rates such that the maximum void dimension in the solid dosage form is in the range from about 2 to 100 microns and the void fraction is in the range of about 5 to 95 percent.

The non-thermosetting polymerized plastics material is preferably a polyol, suitably lactitol, xylitol and sorbitol, erythritol, mannitol, and maltitol. Lactitol is preferred because it is has an ideal melting point, because of its flowability, because it is non-hygroscopic, and because it returns to solid form after melting.

Other substances, for example, polyethylene oxide, can be utilized as the non-thermosetting plastics material. Additional ingredients, such as starches or compounds classified by their dextrose equivalents, such as maltodextrin can be included in the polymer.

The process of the invention produces a novel pharmaceutical dosage form in which the active pharmaceutical agent and the solid excipient are in combination as a homogeneous solid mixture primarily in the form of a rigid microcellular foam. When the foam is formed into tablets or other dosage forms by injection molding, the rigid microcellular foam is enclosed within a skin having a density substantially greater than that of the microcellular foam, but having the same composition as that of said solid mixture.

The homogeneous solid mixture can be made from a composition having a sufficiently high solubility in saliva that a tablet composed of the mixture will dissolve substantially immediately in the mouth upon oral administration. Microcellular foam is particularly well suited for use in flash-dissolve tablets. Its cellular structure promotes quick solution, but it is much less friable than the materials used in conventional flash-dissolve tablets.

The cellular structure of the microcellular foam also enables it to have a low density such that the overall density of the dosage form is substantially less than that of stomach fluids, so that the dosage form is gastro-retentive.

The technique of saturating a mixture of a polymer and an active pharmaceutical agent with a gas, or introducing a supercritical fluid into the mixture, can significantly improve the rate of production of an extrudate for injection molding of pharmaceutical dosage forms. The process makes it possible to achieve desired cell sizes and densities in a continuous process, at a reasonable cost, and with superior quality control.

### BRIEF DESCRIPTION OF THE DRAWINGS

FIG. 1 is a schematic diagram illustrating the process for producing pharmaceutical dosage forms in accordance with the invention;

FIG. 2 is a schematic view of the extruder and mold;

FIG. 3 is a diagram showing a typical mold cavity configuration; and

FIG. 4 is a photograph illustrating a portion of a pharmaceutical dosage form in accordance with the invention.

### DETAILED DESCRIPTION OF THE INVENTION

The invention is directed to production of novel drug/active agent-impregnated microcellular foams, in solid dosage forms such as tablets or caplets. By the adaptation of microcellular foam techniques, used heretofore for producing strong, light weight products such as automotive dashboards and plastic eating utensils, to the manufacture of pharmaceutical dosage forms, it is now possible to take advantage of injection molding or extrusion to produce high quality solid dosage forms that have conventional, time release, or flash-dispersal solution characteristics, and to produce these dosage forms at low cost by forming them continuously over a long time without interruption.

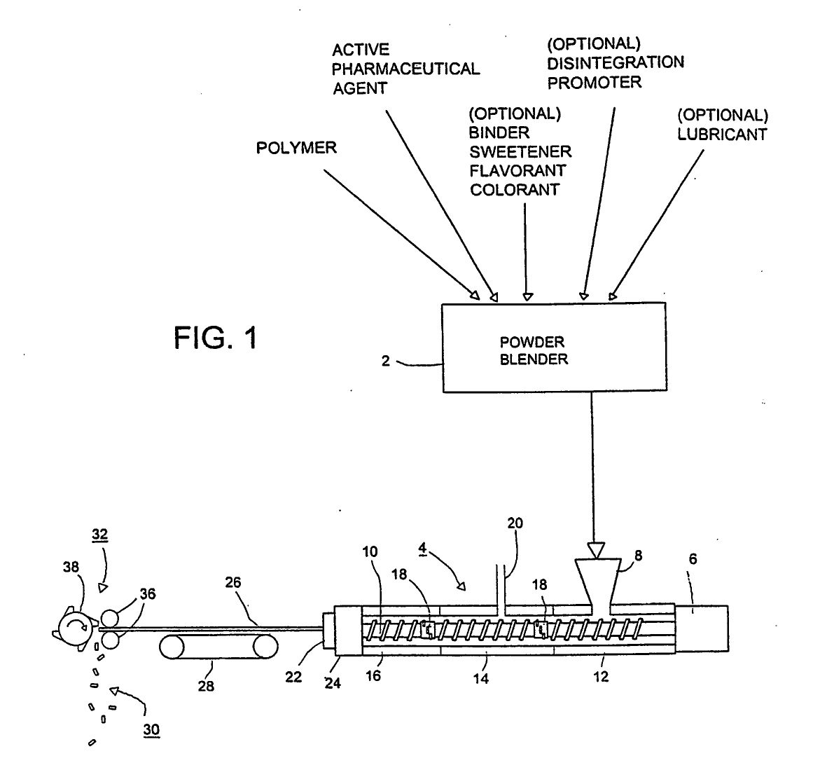

Referring to FIG. 1, as a preliminary step, a pharmaceutically active agent and a polymer are blended in a powder blender 2 and subjected to melt extrusion in a conventional twin-screw extruder 4 having a drive motor 6, a hopper 8 and a pair of screws in side-by-side, meshing relationship, one of which is seen at 10. Heaters 12, 14 and 16 are provided along the extruder 4 to establish separate heated zones. Mixing elements 18 are provided at intervals along the screws in order to ensure homogeneity in the polymer-pharmaceutical agent blend in the extrusion. A liquid injection port 20 is also provided at a location about half way along the length of the barrel of the extruder.

The mixture advanced by the twin screws is extruded through a die 22 having a heater 24. The extruded mixture is preferably in the form of one or more circular cylindrical strands 26, each having a diameter of about 2-3mm. The strand 26 is air-cooled on a strand conveyor 28 and cut into pellets 30, each about 2-3 mm in length, by a strand pelletizer 32 comprising a pair of rollers 36 and a rotating cutter 38.

The proportion of active agent in the mixture is typically between 0.1% and 70%, suitably 10-50%, of the total weight of the mixture. Various additional ingredients, used to control the properties of the product, or of its intermediate forms, may be included. These additional ingredients may be, for example, binders, sweeteners, flavorants, or colorants. The additional ingredients may also be disintegration promoters such as effervescing agents or substances which absorb water and expand. Lubricants to prevent the mixture from adhering to the mold may also be included.

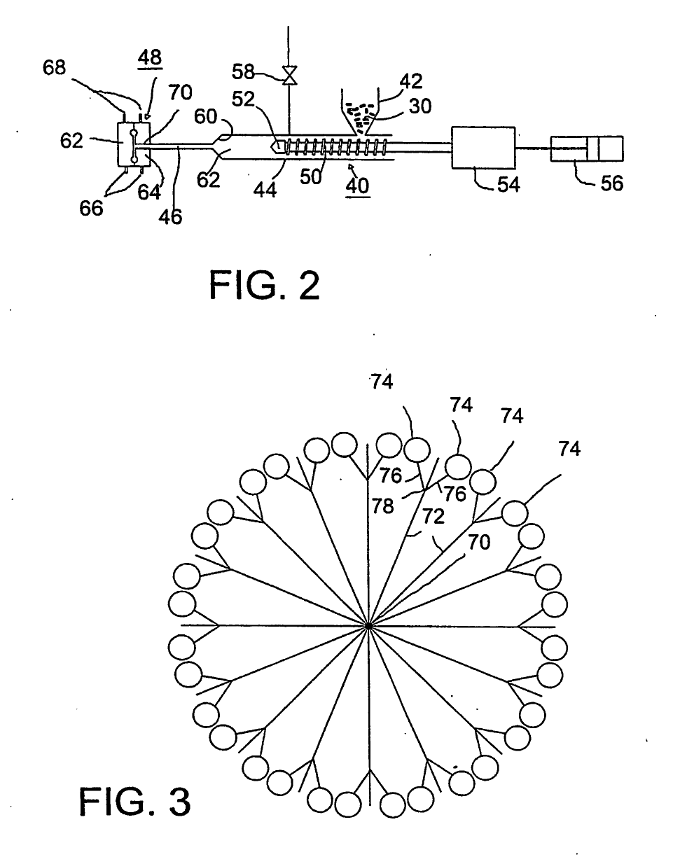

The melt extrusion process results in homogeneous pellets 30, which are delivered to the injection molding machine 40 as shown in FIG. 2. The pellets are introduced into a hopper 42, located near one end of an elongated, hollow barrel 44. A heated nozzle 46, formed at the opposite end of the barrel, is connected to mold 48, which is a multicavity mold. The barrel 44 is heated by an electrical heating coil (not shown) or other suitable heating device in order to melt the pellets after they pass from the hopper into the interior of the barrel. A screw 50 extends longitudinally within barrel 44, and has a one-way valve 52 at its end nearest the nozzle 46. The screw is rotated by a motor 54, and is also reciprocable longitudinally within the barrel by an actuator 56. The screw is shown in its withdrawn position. A valve 58 is provided, through which a gas or SCF can be injected into the interior of the barrel.

In the operation of the injection molding machine, the screw 50 is initially moved forward to a position in which the one-way valve is seated against seat 60, closing off the nozzle 46 The rotation of the screw forces the melted mixture forward while causing the screw itself to move longitudinally in the opposite direction, forming a cushion 62 of melted material in the barrel forward of the one-way valve 52. While the screw is operating, gas, or supercritical fluid, is introduced into the barrel through valve 58. After the cushion is formed, the actuator 56 initiates an injection stroke, pushing the screw 50 toward the nozzle and thereby forcing the cushion of melted material through the nozzle and into the mold 48 during the injection stroke.

The mold 48 is a multicavity mold comprising two mating parts, 62 and 64, which can be separated from each other for removal of the molded dosage forms. Each mold part is cooled by passing a coolant through a coolant inlet port 66 and exhausting coolant through a coolant outlet port 68. The coolant is cycled through a refrigerator/heat exchanger (not shown). The melted mixture, comprising polymer, active pharmaceutical agent, and dissolved gas or SCF, is injected into mold 48 through sprue 70.

In FIG. 3, which illustrates a typical cold runner mold cavity configuration, the radial runners 72 connect the centrally located sprue 70 to the mold cavities 74, which are disposed in a circular pattern. In the configuration shown, each radial runner 72 serves two cavities 74, there being two oblique branches 76 extending respectively to the two cavities from an intermediate point 78 on each radial runner. The connection of the oblique runner branches 76 to the radial runners 72 at intermediate points 78, short of the outer ends of the radial runners, ensures that the melted material delivered through each radial runner will consistently flow into both cavities served by that runner.

Alternatively, a “hot runner” system, known to those skilled in the art, can be used. In such a system, polymer flowing through the nozzle 46 enters heated channels that supply molten polymer to nozzles that feed individual cavities. Each nozzle is also heated to ensure that the polymer remains in a molten condition throughout the entire molding cycle. In this way, material is not wasted, as in the cold runner system, and cycle times are reduced, resulting in a more efficient process. A “valve-gated” nozzle, one having a central rod for shutting off the nozzle outlet, or a “hot-tip” nozzle, where the outlet remains open, may be used. The “valve-gated” nozzle is preferred for the molding of foam tablets, as it will maintain molten material under pressure while the mold is opened for the ejection of molded tablets.

The processing of the mixture in injection molding machine 40 is preferably carried out by injecting a supercritical fluid, such as carbon dioxide or nitrogen, into the melted mixture within barrel 44 of the injection molding machine. At the location at which the fluid is injected, the pressure on the melted mixture is sufficiently high that the fluid remains in its supercritical state, so that the fluid and the melted mixture form a single phase solution. The single phase solution is then injected, by axial movement of the screw 50, into the mold, where the reduction in pressure allows the supercritical fluid to come out of solution in the form of gas bubbles. The gas forms a closed cell foam having a matrix of voids surrounded by a solid lattice. The coolant in the mold limits the expansion of the gas by rapidly solidifying the polymer, thereby keeping the maximum dimension of the voids within in a range of about 2 to 100 microns, a size much smaller than the voids in a conventionally produced foamed polymer.

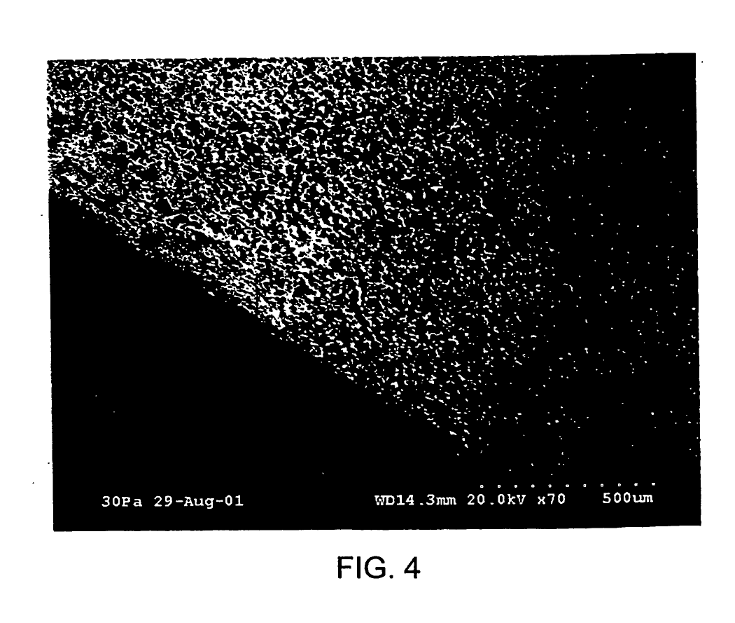

As shown in FIG. 4, the voids have a nearly uniform distribution throughout the foam, and a substantially uniform size, the sizes of almost all of the voids being within a relatively small portion of a preferred range of 10 to 50 microns. The void fraction, that is, the volume of the cells divided by the total volume of the foam, is preferably in the range of about 5% to 95%.

In accordance with a preferred embodiment of the invention, a microcellular foamed material is formed by injection molding in three stages. First a polymer/supercritical fluid mixture is formed. Then, the formation of a single-phase polymer/supercritical fluid solution is completed. Finally, thermodynamic instability is induced in the solution to produce nucleation and expansion of the solution to produce a foamed material having a large number of microscopic voids or cells. Although the process specifically described utilizes supercritical fluids, similar techniques can be used to obtain microcellular materials using gases rather than supercritical fluids.

The polymer/supercritical fluid solution is produced continuously by injecting a supercritical fluid, such as carbon dioxide or nitrogen, into the molten polymer in the barrel 36 of the injection molding machine. The amount of supercritical fluid delivered is preferably metered either by using a positive displacement pump (not shown), or by varying the injection pressure of the supercritical fluid as it passes through a porous material (not shown), which acts to resist the fluid flow. The metered supercritical fluid is then delivered to the extrusion barrel where it is mixed with the molten polymer flowing therein to form a single phase polymer/supercritical fluid mixture.

The supercritical fluid in the mixture then diffuses into the polymer melt to complete the formation of a uniform, single-phase solution of polymer and supercritical fluid. The weight ratio of supercritical fluid to polymer is typically about 10% or more. The maximum amount of a supercritical fluid soluble in a polymer depends on the working pressure and the temperature of the barrel. Using high pressures and/or lower processing temperatures increases the maximum amount of supercritical fluid soluble in the polymer. Therefore, higher pressures and/or lower temperatures are preferable, in order to dissolve the maximum amount of gas, to achieve a high ratio of supercritical fluid to polymer, and in order to achieve high nucleation cell densities.

When the polymer/fluid system, containing a sufficient amount of supercritical fluid, becomes a uniform and homogeneous single-phase solution, the pressure is rapidly reduced to induce thermodynamic instability and to promote a high rate of bubble nucleation in the solution. Typical pressure drop rates used in accordance with the invention to produce foamed pharmaceutical dosage forms are higher than the rates previously used for producing microcellular foamed parts. The pressure drop rate in accordance with the invention preferably exceeds 0.9 GPa/s.

The nucleated polymer/supercritical fluid solution can be supplied either immediately or after a delay, at a selected pressure, to a shaping system such as a die, where expansion and foaming of the solution occurs. In order to prevent the final cell shape from being distorted, the nucleated polymer/supercritical fluid solution can be maintained under pressure within the die until the shaping process has been completed.

By the technique described above, a continuous stream of microcellular, or supermicrocellular polymer is produced. A wide variety of polymers, including but not limited to amorphous and/or semicrystalline polymers, can be used, so long as they are capable of absorbing a gas or a supercritical fluid. Moreover, any gas or supercritical fluid can be used, provided that it is sufficiently soluble in the polymer that is being processed.

Chemical blowing agents may also be used in accordance with the invention, but must be pharmaceutically acceptable, that is, they must meet various guidelines for toxicity, etc. Generally accepted chemical blowing agents for use in the injection molding of PVC, polypropylene, and polyethylene, for example, include, but are not limited to: azodicarbonamides (NH₂—CON═NCO—NH₂, with or without modified substitution products), offered by Uniroyal under the trademark CELOGEN AZ; sulphonyl hydrazines/dinitropentamethylenetetramine/p-toluene sulphonyl semicarbazide; ammonium or sodium bicarbonate (which upon heating will release CO₂). Both ammonium and sodium bicarbonate are USP reagents and can be ingested. Thus they are preferred chemical blowing agents for use in production of pharmaceutically acceptable tablets.

Suitable gas blowing agents for direct injection into the melted polymer include, but are not limited to, chlorofluorocarbons, hydrofluororcarbons, nitrogen, carbon dioxide, argon, and aliphatic hydrocarbons.

The chlorofluorocarbons, CFC-11, CFC-12, used historically to make foamed polystyrene products, but banned in most countries because of their ozone depletion potential, have been replaced with HCFCs and HFCs, which exhibit reduced, or zero, ozone depletion potential. DuPont produces FORMACEL-Z2 (HFC-152a), FORMACEL-S (HCFC-22) and FORMACEL-Z4 (HFC-134A) and Elf Atochem produces a similar selection under the brand name FORANE (HFC-141b and HFC-134a). A preferred chlorofluorocarbon blowing agent for use in accordance with the invention is HFC-134a.

Nitrogen, carbon dioxide, and argon, all of which have been injected into melts of industrial polymers such as polypropylene, polystyrene and polyethylene, etc., to form structural foams, are preferred for use in accordance with the invention, as these gases can be used in the supercritical range, to produce a finer, more uniform, closed cell size.

Examples of aliphatic hydrocarbons which can be utilized as gas blowing agents for direct injection into the melted polymer, are butane, propane, and heptane.

Reaction injection molding (RIM) is also potentially usable to produce microcellular products in accordance with the invention. In reaction injection molding, a polymer mix is heat-activated to initiate a chemical reaction in which a gas evolves, forming bubbles in the melt. For example polyurethane foam is generally produced in this manner. Some polyurethane foams are hydrophilic, can absorb large quantities of water, and can be useful as wound dressings. At present polyurethane is not approved for oral ingestion. However it is contemplated that suitable ingestable, microcellular dosage forms can be produced by reaction injection molding.

The process in accordance with the invention can be used to produce a water-soluble foam product which can be formed into a pledgette. A water-soluble, foam pledgette, suitable for introduction into a nasal passage, can incorporate a desired active agent or agents, for instance suitable antibiotics to treat nosocomial infections in patients or medical staff. The process can also be used to produce water-soluble foam products containing active agents for application to wound dressings. In this case, the active agents can be, for example, mipirocin, the plueromutilins or other topical antibiotics or antiviral agents or co-formulations with other agents, such as silver sulfisalizine. Similarly, the water-soluble foam product can be formed into a suppository or pessary suitable for administration into the rectum or vaginal cavity.

The foam product in accordance with the invention can be utilized as a post-surgical sponge to staunch blood flow and absorb secretions following, for instance, nasal surgery. However unlike conventional, commercially available, post-surgical sponges, which are typically made of insoluble, but swellable, polyvinyl alcohol (PVA), a post-surgical sponge in accordance with the invention can utilize a water soluble polymer containing an active agent intended for absorption into the patient. The post-surgical sponge in accordance with the invention can therefore have not only a fluid-absorbing effect, but also a pharmacological effect.

As mentioned previously, a particularly useful embodiment of this invention is a tablet, preferably a flash-dispersion, or flash-dissolving tablet, formed of a microcellular foamed polymer, such as a polyol or polyethylene oxide, in which an active pharmaceutical composition has been incorporated. Among the advantages of these flash-dispersion formulations are that they are especially suitable for pediatric patients and others who have difficulty in swallowing, its ease of administration, and the ease with which care givers can confirm dosing in the case of institutionalized patients. The microcellular structure of the dosage form ensures good control over the void fraction and enables the manufacturer to maintain the dosage in a given tablet within very close tolerances. The microcellular internal configuration also makes it possible to achieve a relatively high void fraction, which contributes to rapid solution of the tablet, while at the same time producing a tablet having sufficient resistance to breaking up in handling that it can be supplied in conventional bottles rather than in blister packages.

The tablets can be produced by extrusion without injection molding, in which case the dosage can be determined by cutting the extrusion to a desired length. The process of extrusion and cutting has the advantage that the desired dosage levels can be easily changed. Elimination of the injection molding step reduces production time, reduces the cost per tablet, and avoids some environmental concerns about coloring and coating. Preferably, however, the tablet is injection molded, and, unlike the tablet formed by extrusion and cutting, it will have a skin which is more dense than the interior of the tablet, as shown in FIG. 4. The skin contributes to the strength of the tablet, and its resistance to friability, and also makes it possible to print, emboss or engrave information on the tablet in the molding process.

In an alternative embodiment, the pharmaceutical composition can be provided in a non-soluble, acid-stable polymer foam, or an erodable polymer foam. Because of the foam structure, the density of the tablet can be made substantially less than the density of stomach fluids. The lower density dosage form is gastroretentive in that it floats in the stomach fluids, and allows for the leaching of the drug from the foam matrix for gastric delivery, or sustained release gastric delivery.

Various types of final products can be made by the techniques described herein. These include products in the following general categories: flash dispersal products, buccal dosage products, sachet/effervescent products, suppositories or pessaries, and conventional oral tablets.

Flash dispersal products typically provide for delivery of a low dose, high potency drug, preferably containing less than 35 mg of active agent. Suitable active agents for use herein include REQUIP®, AVANDIA®, PAXIL®, and AMERGE®.

In buccal dosage products, also intended for solution in the mouth, it is preferable that the polymer be sufficiently mucoadhesive to coat the buccal/sublingual mucosa. Alternatively, if the coating can be retained in the mouth long enough to allow for drug absorption, and if the drug has a sufficient permeability across mucosa (or an acceptable permeability enhancer is included), buccal delivery is possible. It is preferable that the drug has a high water solubility, and high potency (as it is only possible to deliver a few milligrams by buccal delivery). Taste masking may be needed as well. Buccal delivery has only traditionally been applied to a handful of products, such as nitroglycerin, the ergot alkaloids, nitrates and selegiline.

Water solubility of the active agent is defined by the United States Pharmacoepia. Therefore, active agents which meet the criteria of very soluble, freely soluble, soluble and sparingly soluble as defined therein are encompassed this invention.

The microcellular foam lends itself especially well to sachet products, which are intended to be dissolved in a glass of water, with or without effervescing agents. The foamed structure enhances the solubility of the product. The foam may be granulated and packaged as necessary.

In the case of suppositories and pessaries, the final product can be injection molded to suitable shapes for rectal or vaginal drug delivery.

The process of the invention can, of course, also be used to prepare conventional oral tablets, including immediate release (IR) tablets, sustained release/controlled release (SR/CR) tablets, and even pulsitile release (PR) tablets.

The terms “pharmaceutical agent”, “pharmaceutically acceptable agent”, “medicament”, “active agent” and “drug,” are used interchangeably herein, and include agents having a pharmacological activity in a mammal, preferably a human. The pharmacological activity may be prophylactic or for treatment of a disease. The term is not meant to include agents intended solely for agricultural and/or insecticidal usage or agents intended solely for application to plants and/or soil for other purposes.

The term “tablet,” as used herein, is intended to encompass the elongated forms known as “caplets” as well as other similar dosage forms, including coated dosage forms.

The dosage forms in accordance with the invention may also include additional pharmaceutically acceptable excipients, including but not limited to sweeteners, solubility enhancers, binders, colorants, plasticizers, lubricants, (super)disintegrants, opacifiers, fillers, flavorants, and effervescing agents.

Suitable thermoplastic polymers can be preferably selected from known pharmaceutical excipients. The physico-chemical characteristics of these polymers will dictate the design of the dosage form, such as rapid dissolve, immediate release, delayed release, modified release such as sustained release, or pulsatile release etc.

However, for purposes herein representative examples of thermoplastic polymers suitable for pharmaceutical applications, include, but are not limited to, poly(ethylene oxide), poly(ethylene glycol), especially at higher molecular weights, such as PEG 4000, 6450, 8000, produced by Dow and Union Carbide; polyvinyl alcohol, polyvinyl acetate, polyvinyl-pyrrolidone (PVP, also know as POVIDONE, USP), manufactured by ISP-Plasdone or BASF-Kollidon, primarily Grades with lower K values (K-15, K-25, but also K-30 to K-90); copovidone, polyvinylpyrrolidone/vinyl acetate (PVP/VA) (60:40) (also known as COPOLYVIDONUM, Ph Eur), manufactured by ISP, PLASDONE S-360 or BASF KOLLIDON VA64; hydroxypropylcellulose (HPC), especially at lower molecular weights, e.g., KLUCEL EF and LF grades, available from Aqualon; polyacrylates and its derivatives such as the Eudragit family of polymers available from Rohm Pharma, poly(alpha-hydroxy acids) and its copolymers such poly(caprolactone), poly(lactide-co-glycolide), poly(alpha-aminoacids) and its copolymers, poly(orthoesters), polyphosphazenes, poly(phosphoesters), and polyanhydrides, or mixtures thereof.

Most of these pharmaceutically acceptable polymers are described in detail in the Handbook of Pharmaceutical excipients, published jointly by the American Pharmaceutical association and the Pharmaceutical society of Britain.

Polymeric carriers are divided into three categories: (1)water soluble polymers useful for rapid dissolve and immediate release of active agents, (2) water insoluble polymers useful for controlled release of the active agents; and (3) pH sensitive polymers for pulsatile or targeted release of active agents. It is recognized that combinations of both carriers may be used herein. It is also recognized that several of the polyacrylates are pH dependent for the solubility and may fall into both categories.

Preferably, a water soluble polymer for use herein is hydroxpropylcellulose or polyethylene oxide, such as the brand name POLYOX, or mixtures thereof. It is recognized that these polymers may be used in varying molecular weights, with combinations of molecular weights for one polymer being used, such as 100K, 200K, 300K, 400K, 900K and 2000K. Sentry POLYOX is a water soluble resin which is listed in the NF and have approximate molecular weights from 100K to 900K and 1000K to 7000K, and may be used as 1%, 2% and 5% solutions (depending upon molecular weight).

Additional preferred polymers include povidone, having K values and molecular weight ranges from:

| K value | Mol. wt. |
|---|---|
| 12 | 25 |
| 15 | 8000 |
| 17 | 10,000 |
| 25 | 30,000 |
| 30 | 50,000 |
| 60 | 400K |
| 90 | 1000K |
| 120 | 3000K |

These pharmaceutically acceptable polymers and their derivatives are commercially available and/or be prepared by techniques known in the art. By derivatives it is meant, polymers of varying molecular weight, modification of functional groups of the polymers, or co-polymers of these agents, or mixtures thereof.

Another aspect of the present invention is the use of novel, non-thermoplastic or non-thermosetting excipients (i.e., polyols, starches or maltodextrin), which have been found, when combined with other materials or excipients to create a material that behaves as if it were thermoplastic in the injection molding process. The combination of materials is identified herein as a non-thermosetting polymerized plastic material (nTPM). For instance, while neither lactitol nor maltodextrin are thermoplastic, when blended by hot-melt extrusion, the resultant material can be processed by injection molding as if it were a thermoplastic material. Adjusting the amount of water-soluble excipients (i.e., polyols) in the blends will change the disintegration performance of the material from an immediate release to a more prolonged disintegration. It should be noted, that be adjusting the amount and/or molecular weight of a thermoplastic polymeric carriers (i.e., hydroxypropylcellulose or poly(ethylene oxide)) can effect the disintegration performance of the material as well. In general, higher amounts and/or high molecular weight polymeric carriers will prolong the release performance. Adjusting the levels of water-soluble and polymeric excipients can give a wide spectrum of disintegration from immediate release too much prolonged (i.e., >24 hours) disintegration of the dosage form.

The non-thermosetting polymerized plastic material is a combination of a polyol, and a non-thermosetting or non-thermoplastic polymer, and/or a non-thermosetting or non-thermoplastic modifier.

For purposes herein representative examples of non-thermoplastic polymers suitable for pharmaceutical applications, include, but are not limited to, relatively water soluble polymers such as the cellulose derivatives, such as carboxymethyl cellulose sodium, methyl cellulose, ethylcellulose, hydroxyethylcellulose (HEC), especially at lower molecular weights, such as NATRASOL 250JR or 250LR, available from Aqualon; hydroxypropylmethyl cellulose (HPMC), hydroxypropylmethyl cellulose phthalate, cellulose acetate phthalate, noncrystalline cellulose, starch and its derivatives, and sodium starch glycolate. The thermosetting polymers are typically present in ranges from 2-90%, preferably 5 to 50%. Percentages are in w/w of total dosage form unless otherwise indicated.

In the invention, the non-thermosetting polymeric excipients can be inherently thermoplastic and therefore be readily injection moldable into solid dosage forms.

For purposes herein representative examples of non-thermosetting modifiers suitable for pharmaceutical applications, which in addition to aiding in the production of a non-thermosetting polymerized plastics material also make a more robust dosage form such as by preventing friability and holding the product together, and include carrageenan, especially, lambda type, VISCARIN GP-109NF, available from FMC; polyvinyl alcohol, starches; polyalditol, hydrogenated starch hydrosylate, sodium starch glycolate, maltodextrin, dextrose equivalents, dextrin, and gelatin. The thermosetting modifiers are typically present in ranges from 2-90%, preferably 5 to 50%.

A suitable material which can be processed as non-thermosetting polymerized plastics material is a polyol, such as lactitol, xylitol, sorbitol, erythritol, maltitol, and mannitol, typically in amounts ranging from 5%-70%, preferably 5 to 50%, 5 to 25%. The polyols which can also act as sweeteners, may also impart rapid solubility to the dosage form. As noted previously, lactitol as lactitol monohydrate, USP, is a preferred polyol for use in accordance with the invention.

Non-thermosetting modifiers identified as starches, include but are not limited to pregelatinized Corn Starch, Corn Starch, hydroxyethyl starch, or Waxy maize starch, or mixtures thereof, typically in content ranges from 5-25%. Additional reagents, for use herein are the Polyalditols, (e.g. Innovatol PD30 or PD60: the reducing sugars are <1%); and Hydrogenated starch hydrosylates (ex. Stabilte SD30 and SD60).

Non-thermosetting modifiers identified as maltodextrins, include but are not limited to Maltodextrin, typically in a concentration of 5-50%, classified by DE (detrose equivalent) and have a DE range of 5-18. The lower the DE number the more like starch, which has a DE of about 0. The higher the number the more water soluble corn syrup solids, which have a DE range of 20 to 26. Grades that have been found to be useful are characterized by Maltrin M150 (DE 13-17), Maltrin M180 (DE 16.5-19.5) and Maltrin QD M550 (DE 13-17) from Grain Processing Corporation.

Suitable colorants for use herein can include food grade soluble dyes and insoluble lakes, and are typically present in ranges of about 0.1 to 2%.

Suitable sweeteners can be utilized, in addition to the polyols, such as aspartame, NF, sucralose and saccharin sodium, USP , or mixtures thereof, typically in content ranges from 0.25% to 2%.

Suitable plasticizers, include triacetin, USP, triethyl citrate, FCC, glycerin USP, diethyl phthalate, NF, or tributyl citrate, and mixtures thereof. These liquid plasticizers are typically present in ranges from 1 to 10%.

Suitable lubricants, include food grade glycerol monosterate, stearyl alcohol NF, stearic acid NF, Cab-O-Sil, Syloid, zinc stearate USP, magnesium stearate NF, calcium stearate NF, sodium stearate, cetostrearyl alcohol NF, sodium stearyl fumerate NF, or talc, USP, and mixtures thereof. The lubricant content is typically in the range from 0.1% to 2.5%.

Substances suitable for use as opacifiers/fillers include talc USP, calcium carbonate USP, or kaolin USP, and mixtures thereof. The opacifier/filler content is typically in the range from 0.5 to 2%.

Suitable effervescing agents, include carbonates and bicarbonates of sodium, calcium, or ammonium, along with acids such as malic acid and citric acid, typically in the range from 0.1 to 10%.

Suitable disintegrants and superdisintegrants for use herein include but are not limited to crospovidone, sodium starch glycolate, Eudragit L100-55, sodium carboxymethylcellulose, Ac-di-sol®, carboxymethyl-cellulose, microcrystalline cellulose, and croscarmellose sodium alone or in combination, facilitate the disintegration and solution of the tablet by swelling in the presence of bodily fluids. Disintegrants are typically in the range from 0.1 to 10%.

Suitable binders for use herein include but are not limited to Veegum®, alginates, alginic acid, agar, guar, tragacanth, locust bean, karaya, gelatin, instantly soluble gelatin, carrageenans, and pectin, typically present in an amount of 0.1 to 10%.

It is recognized that certain excipients such as the maltodextrins, starches, hydroxypropylcellulose, hydroxypropylmethyl cellulose, and polyethylene oxides, will also serve as binders and bulking agents in the tablets of this invention. These excipients are either soluble or will absorb water and swell, aiding disintegration of the tablet.

Especially in the production of a flash dispersal tablet, where high water solubility is desired, excipients from some or all of the above categories may be desirable.

For tablets intended to be swallowed, or for controlled or sustained release, excipients from some or all of the above categories may be used, and additional reagents may be desired. The additional reagents, include but are not limited to binders and controlled release (CR) polymers such as, hydroxypropyl-methylcellulose (HMPC), methylcellulose/Na, carboxymethylcellulose, available from Methocels or Aqualon, native or modified starches such as corn starch, wheat starch, rice starch, potato starch, tapioca, and amylose/amylopectin combinations in concentrations of 5%-25%. Maltodextrins may also be useful as a binder or controlled release excipient in concentrations of 5%-50.

The injection molding process as used herein requires the active agent to be stable when subjected to heat, but provides for unique tablet shapes, and release profiles not easily attained by conventional tablet presses.

Suitable pharmaceutically acceptable agents for use in accordance with the invention can be selected from a variety of known classes of drugs including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, parasympathomimetics, parathyroid, calcitonin and biphosphonates, prostaglandins, radiopharmaceuticals, sex hormones (including steroids), anti-allergic agents, stimulants and anorexics, sympathomimetics, thyroid agents, PDE IV inhibitors, CSBP/RK/p38 inhibitors, vasodilators and xanthines.

Preferred pharmaceutically acceptable agents include those intended for oral administration, or by suitable body cavity administration such as rectal or vaginal administration. A description of these classes of drugs and a listing of species within each class can be found in Martindale, The Extra Pharmacopoeia, Twenty-ninth Edition, The Pharmaceutical Press, London, 1989, the disclosure of which is hereby incorporated herein by reference in its entirety. The drug substances contemplated for use herein are commercially available and/or can be prepared by techniques known in the art.

Suitable active ingredients for incorporation into tablets in accordance with the invention may include the many bitter or unpleasant tasting drugs including but not limited to the histamine H2-antagonists, such as, cimetidine, ranitidine, famotidine, nizatidine, etinidine; lupitidine, nifenidine, niperotidine, roxatidine, sulfotidine, tuvatidine and zaltidine; antibiotics, such as penicillin, ampicillin, amoxycillin, and erythromycin; acetaminophen; aspirin; caffeine, dextromethorphan, diphenhydramine, bromopheniramine, chloropheniramine, theophylline, spironolactone, NSAIDS's such as ibuprofen, ketoprofen, naprosyn, and nabumetone; 5HT4 inhibitors, such as granisetron, or ondansetron; seratonin re-uptake inhibitors, such as paroxetine, fluoxetine, and sertraline; vitamins such as ascorbic acid, vitamin A, and vitamin D; dietary minerals and nutrients, such as calcium carbonate, calcium lactate, etc., or combinations thereof.

Where suitable, the above noted active agents, in particular the anti-inflammatory agents, may also be combined with other active therapeutic agents, such as various steroids, decongestants, antihistamines, etc.

Examples of numerous suitable excipients include, but are not limited to the following:

| Chemical Name | Brand Name | Supplier |
|---|---|---|
| Xylitol, NF | Xylisorb | Roquette |
| Hydroxypropyl cellulose, | Klucel | Aqualon |
| Food Grade | | |
| Grade EF: Avg MW- | | |
| 80,000 | | |
| Grade GF: Avg MW- | | |
| 370,000 | | |
| Grade MF: Avg MW- | | |
| 850,000 | | |
| Grade HF: Avg MW- | | |
| 1,150,000 | | |
| Glycerol Monostearate, | | Spectrum |
| NF | | Chem. |
| Croscarmellose Sodium, | AcDiSol | FMC |
| NF | | |
| Copovidone, Ph Eur | Kollidon VA 64 | BASF |
| Erythritol, Food Grade | C*Eridex 16955 | Cerestar |
| Glycerin, USP | | Spectrum |
| | | Chem. |
| Sodium Starch Glycolate, | Explotab | Mendell |
| NF | | |
| Talc, USP | | Spectrum |
| | | Chem. |
| Sorbitol, NF | Neosorb | Roquette |
| Polyethylene Oxide | POLYOX | Dow |
| Grade WSR-N80, | | |
| Avg. MW-200,000 | | |
| Crospovidone, NF | Polyplasdone | ISP |
| Grade XL-10 | | |
| Instantly Soluble | Gelita | Kind & Knox |
| Gelatin | | |
| Type B, MW-3000-9000 | | |
| Methacrylic Acid | Eudragit L100- | Rohm Pharma |
| Copolymer, Type C, | 55 | |
| USP/NF | | |
| Lactitol. Monohydrate, | Lacty M | Purac |
| USP | | |
| Alginic Acid | | Spectrum |
| | | Chem. |
| Sodium Bicarbonate, USP | | Baker |
| Citric Acid, Monohydrate | | Sigma |
| Calcium Carbonate, Light | | Spectrum |
| Powder USP | | Chem. |
| □-Carrageenan | Vascarin | FMC |
| Type GP-109NF | | |
| Magnesium aluminum | VeeGum F | R. T. |
| silicate, Type IB, USP- | | Vanderbilt |
| NF | | |
| Polyethylene glycol, NF | Polyglycol | Dow |
| Type E4500 | | |
| Type E8000 | | |
| Aspartame, NF | | Spectrum |
| | | Chem. |
| Spearmint Concentrate | | International |
| | | Flavors & |
| | | Fragrances |
| Maltodextrin | Maltrin | Grain |
| Maltrin M100, DE 10 | | Processing |
| Maltrin M150, DE 15 | | Corp |
| Microcrystaliine | Emcocel 90 M | Mendell |
| cellulose | | |
| Instantly Soluble Starch | PureCote 3793 | Grain |
| | | Processing |
| | | Corp |
| Pregelatinized starch NF | Starch 1500 | Colorcon |
| Low-substituted | LHPC (LH-11) | Shin Etsu |
| hydroxypropyl cellulose | | |

The extrudability of the mixture and its transformation into pellets is important to the success of the injection molding process. Accordingly, the extrusion process will now be described by reference to a series of examples that are merely illustrative and are not to be construed as a limitation of the scope of the invention. All temperatures are given in degrees Celsius, all solvents are of the highest available purity, and all reactions run under pharmaceutical GMP standards or GLP standards unless otherwise indicated.

In each example, pellets were formed by extrusion of a polymer. The base polymer, binder and other major powdered ingredients (polyol, color, filler, sweeteners, and effervescent agents) were blended in a tumble blender. This blend was then fed into the hopper of a twin-screw extruder where the blend is melted and the screw forces the melt through a 2-3 mm die to make “spaghetti” strands. The strands were air-cooled on a belt conveyer, and then chopped into granules 2-3 mm long by a pelletizer, and fed into a drum. If and when liquid plasticizers or colorants were needed, they were pumped into the polymer melt approximately half-way along the barrel of the extruder. (Alternatively, metering systems can be implemented to feed individual powders, for instance, 4-6 powders, into the extruder without need of a tumble mixer.) Various formulations, and their results are given in the following examples. For blends not containing glycerin as a plasticizer, all pre-mixing was done in a tumble blender (not shown). For those blends containing glycerin, the glycerin is pumped into the barrel of the extruder (through port 20, FIG. 1), using a liquid metering pump (not shown).

In general, for all of the examples, the processing temperatures were between 90° C. and 120° C. in the downstream melting zones and die. Extruder speeds, using an APV Baker MP19 extruder with a 25:1 barrel and 19 mm, co-rotating twin screws, were in the range of 100-200 rpm. Torque, melt pressure at the die and melt temperatures were recorded during processing. When appropriate, extrudate was tested for melt flow rate (MFR) using a capillary rheometer (Kayeness LCR Series) with a die diameter of 0.762 mm and die length of 25.4 mm.

EXAMPLE 1

| | |
|---|---|
| Xylitol | 25% |
| Hydroxypropyl cellulose, Grade EF | 74% |
| Glycerol monostearate | 1% |
| Result: extrusion unsuccessful | |

EXAMPLE 2

| | |
|---|---|
| Xylitol | 25% |
| Hydroxypropyl cellulose, Grade EF | 69% |
| Croscarmellose Sodium | 5% |
| Glycerol monostearate | 1% |

Result: extrusion successful, but not fast-dissolving

EXAMPLE 3

| | |
|---|---|
| Xylitol | 74% |
| Hydroxypropyl cellulose, Grade EF | 20% |
| Croscarmellose Sodium | 5% |
| Glycerol monostearate | 1% |
| Result: extrusion unsuccessful | |

EXAMPLE 4

| | |
|---|---|
| Xylitol | 79% |
| Hydroxypropyl cellulose, Grade EF | 20% |
| Glycerol monostearate | 1% |
| Result: extrusion unsuccessful | |

EXAMPLE 5

| | |
|---|---|
| Xylitol | 74% |
| Copovidone | 20% |
| Croscarmellose Sodium | 5% |
| Glycerol monostearate | 1% |
| Result: extrusion unsuccessful | |

EXAMPLE 6

| | |
|---|---|
| Xylitol | 79% |
| Crospovidone | 20% |
| Glycerol monostearate | 1% |
| Result: extrusion unsuccessful | |

EXAMPLE 7

| | |
|---|---|
| Erythritol | 60% |
| Hydroxypropyl cellulose, Grade EF | 38.5% |
| Glycerol monostearate | 2.5% |
| Result: extrusion unsuccessful | |

Capillary rheometry: MFR@110° C., 9.537 g/10 min

EXAMPLE 8

| | |
|---|---|
| Erythritol | 60% |
| Copovidone | 38.5% |
| Glycerol monostearate | 2.5% |

Result: extrusion somewhat successful,
capillary rheometry: MFR@95° C., 162 g/10 min; Melt
viscosity too low to be viable injection molded material

EXAMPLE 9

| | |
|---|---|
| Erythritol | 60% |
| Hydroxypropyl cellulose, Grade MF | 38.5% |
| Glycerol monostearate | 2.5% |

Result: extrusion unsuccessful, material too viscous

EXAMPLE 10

| | |
|---|---|
| Hydroxypropyl cellulose, Grade EF | 92.5% |
| Glycerin | 5% |
| Glycerol monostearate | 2.5% |

Result: extrusion successful
Capillary rheometry: MFR@130° C., 21.7 g/10 min

EXAMPLE 11

| | |
|---|---|
| Hydroxypropyl cellulose, Grade EF | 87.5% |
| Glycerin | 10% |
| Glycerol monostearate | 2.5% |
| Result: extrusion unsuccessful | |

EXAMPLE 12

| | |
|---|---|
| Hydroxypropyl cellulose, Grade EF | 90.0% |
| Glycerin | 7.5% |
| Glycerol monostearate | 2.5% |
| Result: extrusion successful | |

Capillary rheometry: MFR@130° C., 50.3 g/10 min

EXAMPLE 13

| | |
|---|---|
| Hydroxypropyl cellulose, Grade EF | 91.5% |
| Glycerin | 5% |
| Glycerol monostearate | 2.5% |
| Talc | 1.0% |
| Result: extrusion successful | |

Capillary rheometry: MFR@120° C., 8.391 g/10 min

Using the foam tablet process described above, this formulation was molded into tablets having up to a 50% weight reduction relative to a solid tablet.

EXAMPLE 14

| | |
|---|---|
| Hydroxypropyl cellulose, Grade EF | 53.5% |
| Xylitol | 40.0% |
| Sodium Starch Glycolate, NF | 5.0% |
| Glycerol monostearate | 1.5% |

Result: extrusion unsuccessful, strand too tacky

EXAMPLE 15

| | |
|---|---|
| Hydroxypropyl cellulose, Grade HF | 53.5% |
| Xylitol | 40.0% |
| Sodium Starch Glycolate, NF | 5.0% |
| Glycerol monostearate | 1.5% |

Result: extrusion unsuccessful, insufficient binder,
strand too fragile
Capillary rheometry: viscosity too low for MFR
measurement

EXAMPLE 16

| | |
|---|---|
| Hydroxypropyl cellulose Grade GF | 53.5% |
| Xylitol | 40.0% |
| Sodium Starch Glycolate, NF | 5.0% |
| Glycerol monostearate | 1.5% |
| Result: extrusion somewhat successful | |

Capillary rheometry: MFR@110° C., 107.3 g/10 min

EXAMPLE 17

| | |
|---|---|
| Hydroxypropyl cellulose, Grade EF | 53.5% |
| Sorbitol | 40.0% |
| Sodium Starch Glycolate, NF | 5.0% |
| Glycerol monostearate | 1.5% |

Result: extrusion somewhat successful, strand tacky
Capillary rheometry: viscosity too low for MFR
measurement

EXAMPLE 18

| | |
|---|---|
| Polyethylene oxide (PolyOX, WRS N80) | 70% |
| Sorbitol | 25% |
| Sodium Starch Glycolate, NF | 5% |
| Result: extrusion somewhat successful | |

Capillary rheometry: MFR too temperature dependent to be
useful

EXAMPLE 19

| | |
|---|---|
| Polyethylene oxide (PolyOX, WRS N80) | 45% |
| Sorbitol | 50% |
| Sodium Starch Glycolate, NF | 5% |
| Result: extrusion somewhat successful | |

Capillary rheometry: viscosity too high for MFR
measurement

EXAMPLE 20

| | |
|---|---|
| Polyethylene oxide (PolyOX, WRS N80) | 38.8% |
| Sorbitol | 49.6% |
| Crospovidone | 5.5% |
| Instantly Soluble Gelatin | 5.5% |
| Glycerol monostearate | 1.1% |

Result: extrusion successful but strand needed to cool
on bench
Capillary rheometry: MFR@90° C., 7.934 g/10 min
MFR@95° C., 163.381 g/10 min (MFR too temperature sensitive
to be viable)

EXAMPLE 21

| | |
|---|---|
| Hydroxypropyl cellulose, Grade EF | 49% |
| Sorbitol | 40% |
| Crospovidone | 5% |
| Instantly Soluble Gelatin | 5% |
| Glycerol monostearate | 1% |

Result: extrusion unsuccessful

EXAMPLE 22

| | |
|---|---|
| Hydroxypropyl cellulose, Grade GF | 49% |
| Sorbitol | 40% |
| Crospovidone | 5% |
| Instantly Soluble Gelatin | 5% |
| Glycerol monostearate | 1% |
| Result: extrusion unsuccessful | |

EXAMPLE 23

| | |
|---|---|
| Polyethylene oxide (PolyOX, WRS N80) | 40% |
| Sorbitol | 49% |
| Crospovidone | 5% |
| Eudragit L100-55 | 5% |
| Glycerol monostearate | 1% |
| Result: extrusion poor | |

Capillary rheometry: MFR@90° C., 22.328 g/10 min

EXAMPLE 24

| | |
|---|---|
| Polyethylene oxide (PolyOX, WRS N80) | 40% |
| Lactitol | 49% |
| Crospovidone | 5% |
| Eudragit L100-55 | 5% |
| Glycerol monostearate | 1% |
| Result: extrusion acceptable | |

Capillary rheometry: MFR@115° C., 10.870 g/10 min

EXAMPLE 25

| | |
|---|---|
| Polyethylene oxide (PolyOX, WRS N80) | 40% |
| Lactitol | 49% |
| Crospovidone | 5% |
| Alginic Acid | 5% |
| Glycerol monostearate | 1% |
| Result: extrusion acceptable | |

Capillary rheometry: MFR@110° C., 1.726 g/10 min

EXAMPLE 26

| | |
|---|---|
| Polyethylene oxide (PolyOX, WRS N80) | 40% |
| Lactitol | 45% |
| Crospovidone | 5% |
| Alginic Acid | 5% |
| Sodium bicarbonate | 4% |
| Glycerol monostearate | 1% |
| Result: extrusion acceptable | |

Capillary rheometry: MFR@110° C. 1.686 g/10 min

EXAMPLE 27

| | |
|---|---|
| Polyethylene oxide (PolyOX, WRS N80) | 30% |
| Lactitol | 59% |
| Crospovidone | 5% |
| Eudragit L100-55 | 5% |
| Glycerol monostearate | 1% |
| Result: extrusion acceptable | |

Capillary rheometry: MFR@110° C., 3.106 g/10 min

EXAMPLE 28

| | |
|---|---|
| Polyethylene oxide (PolyOX, WRS N80) | 20% |
| Lactitol | 69% |
| Crospovidone | 5% |
| Eudragit L100-55 | 5% |
| Glycerol monostearate | 1% |
| Result: extrusion unacceptable | |

Capillary rheometry: MFR@110° C., 10.679 g/10 min

EXAMPLE 29

| | |
|---|---|
| Polyethylene oxide (PolyOX, WRS N80) | 30% |
| Lactitol | 62% |
| Crospovidone | 2.5% |
| Citric Acid | 2.5% |
| Calcium bicarbonate | 2.5% |
| Glycerol monostearate | 0.5% |
| Result: extrusion unacceptable | |

Capillary rheometry: MFR@105° C., 8.713 g/10 min

EXAMPLE 30

| | |
|---|---|
| Polyethylene oxide (PolyOX, WRS N80) | 40% |
| Lactitol | 49% |
| Crospovidone | 5% |
| λ-Carrageenan | 5% |
| Glycerol monostearate | 1% |
| Result: extrusion acceptable | |

Capillary rheometry: MFR@110° C., 4.143 g/10 min

EXAMPLE 31

| | |
|---|---|
| Polyethylene oxide (PolyOX, WRS N80) | 15% |
| Lactitol | 65% |
| Citric Acid | 5% |
| Calcium carbonate | 5% |
| λ-Carrageenan | 10% |

Result: extrusion unacceptable, insufficient binder
Capillary rheometry: MFR@105° C., 2.617 g/10 min

EXAMPLE 32

| | |
|---|---|
| Polyethylene oxide (PolyOX, WRS N80) | 15% |
| Lactitol | 55% |
| Sorbitol | 10% |
| Citric Acid | 5% |
| Calcium carbonate | 5% |
| λ-Carrageenan | 10% |

Result: extrusion unacceptable, insufficient binder

EXAMPLE 33

| | |
|---|---|
| Polyethylene oxide (PolyOX, WRS N80) | 25% |
| Lactitol | 60% |
| Citric Acid | 5% |
| Calcium carbonate | 5% |
| λ-Carrageenan | 5% |
| Result: extrusion somewhat acceptable | |

Capillary rheometry: MFR@105° C., 6.571 g/10 min

EXAMPLE 34

| | |
|---|---|
| Polyethylene oxide (PolyOX, WRS N80) | 25% |
| Lactitol | 60% |
| Citric Acid | 5% |
| Sodium bicarbonate | 5% |
| λ-Carrageenan | 5% |

Result: extrusion poor, sodium bicarbonate “volatile”,
foaming strand

EXAMPLE 35

| | |
|---|---|
| Polyethylene oxide (PolyOX, WRS N80) | 30% |
| Lactitol | 50% |
| Citric Acid | 5% |
| Calcium Carbonate | 9.5% |
| VeeGum F | 5% |
| Glycerol Monostearate | 0.5% |
| Result: extruded well at up to 2 kg/hr | |

Capillary rheometry: MFR@110° C., 0.207 g/10 min, very
stiff at this temperature

EXAMPLE 36

| | |
|---|---|
| Polyethylene oxide (PolyOX, WRS N80) | 30% |
| Lactitol | 50% |
| Citric Acid | 5% |
| Calcium Carbonate | 9.5% |
| Crospovidone | 5% |
| Glycerol Monostearate | 0.5% |
| Result: extruded well at up to 2 kg/hr | |

Capillary rheometry: MFR@115° C., 0.060 g/10 min, very
stiff at this temperature

EXAMPLE 37

| | |
|---|---|
| Polyethylene oxide (PolyOX, WRS N80) | 30% |
| Lactitol | 50% |
| Citric Acid | 5% |
| Calcium Carbonate | 9.5% |
| Eudragit L100-55 | 5% |
| Glycerol Monostearate | 0.5% |
| Result: extruded well at up to 2 kg/hr | |

Capillary rheometry: MFR@110° C., 3.068 g/10 min

EXAMPLE 38

| | |
|---|---|
| Polyethylene oxide (PolyOX, WRS N80) | 25% |
| Polyethylene glycol E8000 | 5% |
| Lactitol | 50% |
| Citric Acid | 5% |
| Calcium Carbonate | 9.5% |
| Eudragit L100-55 | 5% |
| Glycerol Monostearate | 0.5% |
| Result: extruded well at up to 2 kg/hr | |

Capillary rheometry: MFR@110° C., 1.719 g/10 min

EXAMPLE 39

| | |
|---|---|
| Polyethylene oxide (PolyOX, WRS N80) | 24.45% |
| Polyethylene glycol E4500 | 5% |
| Lactitol | 50% |
| Citric Acid | 5% |
| Calcium Carbonate | 9.5% |
| Eudragit L100-55 | 5% |
| Glycerol Monostearate | 0.5% |
| Aspartame | 0.5% |
| Spearmint Concentrate | 0.05% |
| Result: extruded well at 1.5 kg/hr | |

Capillary rheometry: MFR@110 C., 0.685 g/10 min

EXAMPLE 40

| | |
|---|---|
| Polyethylene oxide (PolyOX, WRS N80) | 24.45% |
| Polyethylene glycol E4500 | 5% |
| Lactitol | 50% |
| Citric Acid | 5% |
| Calcium Carbonate | 9.5% |
| Eudragit L100-55 | 5% |
| Glycerol Monostearate | 0.5% |
| Aspartame | 0.5% |
| Spearmint Concentrate | 0.05% |
| Result: extruded well at 1.5 kg/hr | |

14 kg of this blend were extruded for trial, and the
extruded material was molded into tablets using the
foam tablet process described above.

Capillary rheometry: MFR@105° C., 6.575 g/10 min, MFR@110° C., 7.204 g/10 min. Up to a 60% weight reduction relative to a solid tablet was achieved.

EXAMPLE 41

| | |
|---|---|
| Polyethylene oxide (PolyOX, WRS N80) | 19.45% |
| Polyethylene glycol E4500 | 10% |
| Lactitol | 50% |
| Citric Acid | 5% |
| Calcium Carbonate | 9.5% |
| Eudragit L100-55 | 5% |
| Glycerol Monostearate | 0.5% |
| Aspartame | 0.5% |
| Spearmint Concentrate | 0.05% |

Result: strand broke readily when extruded, not a viable
formulation

EXAMPLE 42

| | |
|---|---|
| Lactitol | 25% |
| Maltodextrin (Maltrin M100) | 70% |
| Sodium Starch Glycolate | 5% |

Result: starch content too high, pressure exceeded
maximum

EXAMPLE 43

| | |
|---|---|
| Lactitol | 45% |
| Maltodextrin (Maltrin M100) | 50% |
| Sodium Starch Glycolate | 5% |

Result: could be extruded at 2 kg/hr but brittle
Capillary rheometry: MFR@110° C., 41.474 g/10 min

EXAMPLE 44

| | |
|---|---|
| Lactitol | 50% |
| Maltodextrin (Maltrin M150) | 45% |
| Sodium Starch Glycolate | 5% |
| Result: extruded well at 2 kg/hr | |

Capillary rheometry: MFR@110° C., 37.734 g/10 min

EXAMPLE 45

| | |
|---|---|
| Lactitol | 50% |
| Microcrystalline cellulose (Emcocel 90M) | 45% |
| Sodium Starch Glycolate | 5% |

Result: extruded poorly, even at 0.5 kg/hr, too viscous

EXAMPLE 46

| | |
|---|---|
| Lactitol | 50% |
| Maltodextrin (Maltrin M150) | 20% |
| Sodium Starch Glycolate | 25% |

Result: extruded poorly, material too thin to pelletize

EXAMPLE 47

| | |
|---|---|
| Lactitol | 50% |
| Mannitol | 20% |
| Maltodextrin (Maltrin M150) | 20% |
| Instantly Soluble Starch | 5% |
| Sodium Starch Glycolate | 5% |

Result: extruded at 2 kg/hr but the strand was very
thin, did not pelletize well, melt viscosity is very
low; too low to be injection moldable; no MFR could be
calculated.

EXAMPLE 48

| | |
|---|---|
| Lactitol | 50% |
| Mannitol | 25% |
| Instantly Soluble Starch | 15% |
| Sodium Starch Glycolate | 10% |

Result: extruded at 2 kg/hr but the strand was very
thin, did not pelletize well, melt viscosity is very low
Capillary rheometry: MFR@110° C., 119.168 g/10 min

EXAMPLE 49

| | |
|---|---|
| Lactitol | 40% |
| Maltodextrin (Maltrin M150) | 50% |
| Sodium Starch Glycolate | 10% |

Result: extruded very well at 2 kg/hour
Capillary rheometry: MFR@110° C., 12.497 g/10 min

EXAMPLE 50

| | |
|---|---|
| Lactitol | 40% |
| Maltodextrin (Maltrin M150) | 50% |
| VeeGum F | 10% |

Result: extruded very well at 2 kg/hour
Capillary rheometry: MFR@110° C., 13.646 g/10 min

EXAMPLE 51

| | |
|---|---|
| Lactitol | 40% |
| Maltodextrin (Maltrin M150) | 50% |
| AcDiSol | 10% |

Result: extruded very well at 2 kg/hour
Capillary rheometry: MFR@110° C., 15.312 g/10 min

EXAMPLE 52

| | |
|---|---|
| Lactitol | 40% |
| Maltodextrin (Maltrin M150) | 50% |
| Crospovidone | 10% |

Result: extruded very well at 2 kg/hour
Capillary rheometry: 8.995 g/10 min

EXAMPLE 53

| | |
|---|---|
| Lactitol | 40% |
| Maltodextrin (Maltrin M150) | 50% |
| Eudragit L100-55 | 10% |

Result: extruded very well at 2 kg/hour
Capillary rheometry: MFR@110° C., 11.722 g/10 min

EXAMPLE 54

| | |
|---|---|
| Lactitol | 40% |
| Maltodextrin (Maltrin M150) | 50% |
| Eudragit L100-55 | 5% |
| Crospovidone | 5% |

Result: extruded very wall at 2 kg/hour
Capillary rheometry: MFR@115° C., 12.893 g/10 min

EXAMPLE 55

| | |
|---|---|
| Lactitol | 45% |
| Maltodextrin (Maltrin M150) | 40% |
| Pregelatinized Starch NF (Starch 1500) | 5% |
| Crospovidone | 10% |
| Result: extruded very well at 2 kg/hour | |

Capillary rheometry: MFR@110° C., 6.239 g/10 min

EXAMPLE 56

| | |
|---|---|
| Lactitol | 50% |
| Maltodextrin (Maltrin M150) | 30% |
| Pregelatinized Starch NF (Starch 1500) | 10% |
| Crospovidone | 10% |
| Result: extruded well at 2 kg/hour | |

Capillary rheometry: MFR@110° C., 8.075 g/10 min

EXAMPLE 57

| | |
|---|---|
| Lactitol | 45% |
| Maltodextrin (Maltrin M150) | 40% |
| Pregelatinized Starch NF (Starch 1500) | 5% |
| Crospovidone | 5% |
| Eudragit L100-55 | 5% |
| Result: extruded well at 2 kg/hour | |

Capillary rheometry: MFR@110° C., 13.879 g/10 min

EXAMPLE 58

| | |
|---|---|
| Lactitol | 65% |
| Pregelatinized Starch NF (Starch 1500) | 15% |
| Crospovidone | 10% |
| Eudragit L100-55 | 10% |

Result: marginal process at 2 kg/hour, pelletized poorly
with large amount of powder

EXAMPLE 59

| | |
|---|---|
| Lactitol | 60% |
| Crospovidone | 20% |
| Eudragit L100-55 | 20% |

Result: marginal process at 2 kg/hour, insufficient
binder

EXAMPLE 60

| | |
|---|---|
| Lactitol | 40% |
| Calcium carbonate, Light Powder USP | 20% |
| Crospovidone | 20% |
| Eudragit L100-55 | 20% |

Result: marginal process at 1 kg/hour, strand very
fragile

EXAMPLE 61

| | |
|---|---|
| Lactitol | 50% |
| Erythritol | 20% |
| Maltodextrin (Maltrin M150) | 25% |
| Sodium Starch Glycolate | 5% |

Result: processing temperature to form strand very low,
˜70° C., strand required extra cooling time to pelletize.

EXAMPLE 62

| | |
|---|---|
| Lactitol | 65% |
| Maltodextrin (Maltrin M150) | 5% |
| Pregelatinized Starch NF (Starch 1500) | 15% |
| Crospovidone | 7.5% |
| Eudragit L100-55 | 7.5% |

Result: extruded at 2 kg/hour, pelletized poorly with
large amount of powder

EXAMPLE 63

| | |
|---|---|
| Lactitol | 70% |
| Pregelatinized Starch NF (Starch 1500) | 15% |
| Crospovidone | 7.5% |
| Eudragit L100-55 | 7.5% |

Result: extruded at 2 kg/hour, pelletized poorly with
large amount of powder

EXAMPLE 64

| | |
|---|---|
| Lactitol | 65% |
| Erythritol | 5% |
| Pregelatinized Starch NF (Starch 1500) | 15% |
| Crospovidone | 7.5% |
| Eudragit L100-55 | 7.5% |

Result: extruded at 2 kg/hour, pelletized poorly with
large amount of powder

EXAMPLE 65

| | |
|---|---|
| Lactitol | 60% |
| Erythritol | 10% |
| Pregelatinized Starch NF (Starch 1500) | 15% |
| Crospovidone | 7.5% |
| Eudragit L100-55 | 7.5% |

Result: extruded at 2 kg/hour, but strand thinned and
required extra cooling time, pelletized poorly with
large amount of powder

EXAMPLE 66

| | |
|---|---|
| Lactitol | 55% |
| Maltodextrin (Maltrin QD550) | 40% |
| Eudragit L100-55 | 5% |
| Crospovidone | 5% |

Result: extruded very well at 2 kg/hour
Capillary rheometry: MFR@110° C., 18.849 g/10 min

EXAMPLE 67

| | |
|---|---|
| Lactitol | 40% |
| Maltodextrin (Maltrin M180) | 50% |
| Eudragit L100-55 | 5% |
| Crospovidone | 5% |

Result: extruded very well at 2 kg/hour
Capillary rheometry: MFR@110° C., 18.877 g/10 min

EXAMPLE 68

| | |
|---|---|
| Lactitol | 40% |
| Maltodextrin (Maltrin M150) | 45% |
| Eudragit L100-55 | 7.5% |
| Crospovidone | 7.5% |

Result: extruded very well at 2 kg/hour
Capillary rheometry: MFR@115° C., 9.103 g/10 min

EXAMPLE 69

| | |
|---|---|
| Lactitol | 40% |
| Maltodextrin (Maltrin M150) | 45% |
| Eudragit L100-55 | 7.5% |
| Low-substituted hydroxypropyl cellulose | 7.5% |

Result: extruded well at 1.5 kg/hour but strand was soft
Capillary rheometry: MFR@110° C., 13.076 g/10 min

EXAMPLE 70

| | |
|---|---|
| Lactitol | 40% |
| Maltodextrin (Maltrin QD550) | 50% |
| Eudragit L100-55 | 5% |
| Crospovidone | 5% |

Result: extruded well at 2 kg/hour but pelletizing was difficult at times
Capillary rheometry: MFR@110° C., 14.872 g/10 min

EXAMPLE 71

| | |
|---|---|
| Lactitol | 40% |
| Maltodextrin (Maltrin QD550) | 45.5% |
| Eudragit L100-55 | 5% |
| Crospovidone | 7.5% |
| Talc, USP | 2% |

Result: extruded very well at 2 kg/hour
Capillary rheometry: MFR@110° C., 14.908 g/10 min

EXAMPLE 72

| | |
|---|---|
| Lactitol | 40% |
| Maltodextrin (Maltrin QD550) | 43% |
| Eudragit L100-55 | 5% |
| Crospovidone | 10% |
| Talc, USP | 2% |

Result: extruded very well at 2 kg/hour
Capillary rheometry: MFR@110° C., 8.968 g/10 min

EXAMPLE 73

| | |
|---|---|
| Lactitol | 40% |
| Maltodextrin (Maltrin QD550) | 45.5% |
| Eudragit L100-55 | 5% |
| Crospovidone | 7.5% |
| Glycerol Monostearate | 2% |

Result: extruded very well at 2 kg/hour
Capillary rheometry: MFR@110° C., 41.569 g/10 min

EXAMPLE 74

| | |
|---|---|
| Rosiglitazone maleate (anhydrous) | 0.96% |
| Lactitol | 40% |
| Maltodextrin (Maltrin QD550) | 44.55% |
| Eudragit L100-55 | 5% |
| Crospovidone | 7.5% |
| Talc, USP | 2% |

Result: extruded very well at 2 kg/hour
Capillary rheometry: MFR@105° C., 8.868 g/10 min
MFR@110° C., 14.251 g/10 min
Injection molding of blend attempted using mold in FIG. 3. Solid
tablets ejected but runner remained with mold, preventing automatic
operation of the injection molding machine.

EXAMPLE 75

| | |
|---|---|
| Hydroxypropyl cellulose, Grade EF | 93% |
| Glycerin | 4% |
| Glycerol monostearate | 2% |
| Talc | 1% |

Comment: extrusion successful
Capillary rheometry: MFR@120° C., 6.419 g/10 min
Material was successfully injection molded into solid forms.

EXAMPLE 76

| | |
|---|---|
| Carvedilol ® | 5.15% |
| Hydroxypropyl cellulose, Grade EF | 88.85% |
| Glycerin | 4.00% |
| Glycerol monostearate | 2.00% |

Comment: extrusion successful
Capillary rheometry: MFR@120° C., 21.027 g/10 min
Material was successfully injection molded into solid forms.

EXAMPLE 77

| | |
|---|---|
| Carvedilol ® | 5.15% |
| Hydroxypropyl cellulose, Grade EF | 92.85% |
| Glycerol monostearate | 2.00% |

Comment: extrusion successful.
Capillary rheometry: MFR@120° C., 2.736 g/10 min and @125° C.,
5.319 g/10 min
Material was successfully injection molded into solid forms.

EXAMPLE 78

| | |
|---|---|
| Carvedilol ® | 5.15% |
| Hydroxypropyl cellulose, Grade EF | 92.85% |
| Magnesium stearate | 2.00% |
| Comment: extrusion successful | |

Capillary rheometry: MFR@120° C., 6.617 g/10 min
Material was successfully injection molded into solid
forms.

EXAMPLE 79

| | |
|---|---|
| Carvedilol ® | 5.15% |
| Hydroxypropyl cellulose, Grade EF | 92.85% |
| Talc | 2.00% |
| Comment: extrusion successful | |

Capillary rheometry: MFR@120° C., 8.016 g/10 min
Material injection molded poorly.

The inclusion of a polyol (preferably lactitol) in the above examples serves two purposes. First, it is a water-soluble excipient that facilitates disintegration and solution of a flash-dissolve, immediate release tablet. Second, at elevated temperatures, it plasticizes the blend, allowing for extrusion and injection molding.

In general, the process temperature was no higher than 120° C., preferably less than 110° C., and optimally 100° C. or less. The time the polymer blend is exposed to this elevated temperature is no more than about two minutes. In this way potential thermal degradation can be minimized.

In general, blends having an MFR between 5 g/10 minutes and 20 g/10 minutes at the temperature setting for injection molding (i.e., <120° C.) will have a melt viscosity that will allow the material to be injection molded.

Glidants, (i.e., talc, USP, and glycerol monostearate) may be needed in the formulation to prevent tablets from sticking to the mold.

Pellets formed by the melt extrusion process depicted in FIG. 1 were fed into the hopper of an injection molding machine as depicted in FIG. 2, and melted in the barrel. Using the process described in U.S. Pat. Nos. 5,334,356 and 6,051,174, and published International patent applications WO 98/08667 and WO 99/32544, supercritical N₂ was injected into the melted polymer in the injection molding machine. The pressure and temperature were controlled to ensure the supercritical fluid (SCF) formed a single phase with the polymer. The operation of the screw in the molding machine caused a cushion of melted polymer to form at the injection end of the barrel. With the mold closed, the polymer was rapidly forced into the mold by driving the screw forward. Air in the mold was forced out during the injection stroke and the mold cavity completely filled with polymer. When the pressure was reduced in the mold, the gas came out of solution to form microscopic bubbles in the polymer. The mold was chilled, allowing the polymer to “freeze” into tablet shape. The mold was then opened, and ejection pins popped the resultant tablets out of the mold, depositing them into a drum.

A preferred formulation for about 20 kg of a polymer blend to use in this process with an active agent is

| | |
|---|---|
| Hydroxypropylcellulose, Grade EF, MW ˜30,000 | 91.5% |
| Glycerin (as plasticizer) | 5.0% |
| Glycerol monostearate | 2.5% |
| Talc (nucleating agent for foam) | 1.0% |

The invention makes it possible to foam tablets, via an injection molding process, with an approximately 50% weight reduction relative to a solid tablet, of pharmaceutically acceptable polymers, to package the tablets in bottles or other conventional tablet containers instead of molding them in the blister packages in which they are to be sold, and to shape the tablets in any of a broad variety of possible shapes. Once the injection molding machine is stabilized, the process may be run with very little operator involvement, around the clock, producing a very homogeneous product.

By utilization of less soluble pharmaceutically acceptable polymers in the injection molding of tablets, swallowable tablets having varying release characteristics similar to conventional immediate release or controlled release tablets may be produced.

The injection molding of tablets (especially flash-release tablets) significantly reduces the complexity of the pharmaceutical manufacturing process. The injection molding process of this invention preferably utilizes a single excipient feed (pellets extruded from a preceding extrusion process producing a homogenous intermediate), and can be carried out using a single fully-automated injection molding press designed for continuous (24 hour, 7 day) operation.

The novel dosage forms of this invention, based upon a water soluble foam, provide for unique drug delivery possibilities.

Various modifications can be made in the formulations and processes described herein. For example, although the preferred process utilizes supercritical N₂ or CO₂ injection, it is possible to produce suitable microcellular foamed dosage forms by injection of N₂ or CO₂ in gaseous form under pressure into the polymer melt, or to utilize a chemical blowing agent or reaction injection molding. Similarly, whereas in the preferred embodiment, the polymer resin is formulated with the active agent already incorporated into it, the active agent can be introduced in other ways, for example, it can be injected into the melt in the extruder, or where possible, dissolved in, and injected along with, the supercritical fluid.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims.

Without further elaboration, it is believed that one skilled in the are can, using the preceding description, utilize the present invention to its fullest extent. Therefore the Examples herein are to be construed as merely illustrative and not a limitation of the scope of the invention in any way. The embodiments of the invention in which an exclusive property or privilege is claimed are defined as follows.

1. A pharmaceutical dosage form suitable for oral administration comprising a molded microcellular polymeric material and a pharmaceutically acceptable active agent.

2. The pharmaceutical dosage form according to claim 1 wherein the molded microcellular polymeric material is a non-thermosetting polymerized plastics material.

3. The pharmaceutical dosage form according to claim 2 wherein the non-thermosetting polymerized plastics material contains at least one polyol, and at least one non-thermosetting modifier, and/or a non-thermosetting polymer.

4. The pharmaceutical dosage form according to claim 3 wherein the non-thermosetting polymerized plastics material contains at least one polyol, and at least one non-thermosetting modifier.

5. The pharmaceutical dosage form according to claim 3 wherein the polyol is lactitol, xylitol, sorbitol, maltitol, or mannitol, or combinations thereof.

6. The pharmaceutical dosage form according to claim 3 wherein the non-thermosetting modifier is a starch, maltodextrin, a dextrose equivalent, polyalditol a hydrogenated starch hydrosylate, or a mixture thereof.

7. The pharmaceutical dosage form according to claim 6 wherein the starch is pregelatinized corn starch, corn starch, potato starch, rice starch, hydroxyethyl starch, wheat starch, tapioca starch, or waxy maize starch, or mixtures thereof.

8. The pharmaceutical dosage form according to claim 6 wherein the non-thermosetting modifier is a maltodextrin.

9. The pharmaceutical dosage form according to claim 3 wherein the non-thermosetting polymer is carboxymethyl cellulose sodium, methyl cellulose, ethylcellulose, hydroxyethylcellulose (HEC), hydroxypropylmethyl cellulose (HPMC), hydroxypropylmethyl cellulose phthalate, cellulose acetate phthalate, noncrystalline cellulose, starch and its derivatives, and sodium starch glycolate or mixtures thereof.

10. The pharmaceutical dosage form according to claim 1 which optionally further comprises a sweetener, a disintegrant, a binder, a lubricant, or an opacifier.

11. The pharmaceutical dosage form according to claim 10 wherein the disintegrant is croscarmellose sodium, sodium starch glycolate, sodium carboxymethyl-cellulose, Ac-di-sol®, carboxymethyl-cellulose, veegum, an alginate, agar, guar, tragacanth, locust bean, karaya, pectin, or crospovidone.

12. The pharmaceutical dosage form according to claim 10 wherein the lubricant is glycerol monosterate, stearyl alcohol NF, stearic acid NF, Cab-O-Sil, Syloid, zinc stearate USP, magnesium stearate NF, calcium stearate NF, sodium stearate, cetostrearyl alcohol NF, sodium stearyl fumerate NF, or talc.

13. The pharmaceutical dosage form according to claim 10 wherein the opacifiers is talc USP, calcium carbonate USP, or kaolin USP.

14. The pharmaceutical dosage form according to claim 1 wherein the pharmaceutically acceptable active agent is selected from an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antithyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, and a vasodilator.

15. The pharmaceutical dosage form according to claim 1 wherein the molded microcellular polymeric material is a thermoplastic polymer.

16. The pharmaceutical dosage form according to claim 15 wherein the thermoplastic polymer is polyethylene oxide, hydroxypropylcellulose, polyethylene glycol, polyvinyl pyrrolidone, copovidone, or povidone or mixtures thereof.

17. The pharmaceutical,dosage form according to claim 16 wherein the polymer is polyethylene oxide, hydroxypropylcellulose, or a mixture thereof.

18. The pharmaceutical dosage form according to claim 15 which further comprises a non-thermosetting polymerized plastics material.

19. The pharmaceutical dosage form according to claim 18 wherein the non-thermosetting polymerized plastics material contains at least one polyol, and at least one non-thermosetting modifier, and/or a non-thermosetting polymer.

20. The pharmaceutical dosage form according claim 1 wherein the microcellular polymeric material is a closed cell foam.

21. A pharmaceutical dosage form comprising: a rigid microcellular foam consisting of a solid excipient having voids of substantially uniform size with a maximum void dimension in the range from about 2 to 100 microns and a void fraction in the range of about 5 to 95 percent, the solid excipient comprising a non-thermosetting polymerized plastic material and an active pharmaceutical agent combined in a homogeneous solid mixture.

22. The pharmaceutical dosage form according to claim 21 wherein the non-thermosetting polymerized plastics material contains at least one polyol, and at least one non-thermosetting modifier, or non-thermosetting polymer.

23. The pharmaceutical dosage form according to claim 21 wherein the polyol is lactitol, xylitol, sorbitol, maltitol, or mannitol, or combinations thereof.

24. The pharmaceutical dosage form according to claim 21 wherein the non-thermosetting modifier is a starch, maltodextrin, a dextrose equivalent, polyalditol a hydrogenated starch hydrosylate, or a mixture thereof.

25. The pharmaceutical dosage form according to claim 24 wherein the starch is pregelatinized Corn Starch, Corn Starch, Potato starch, Rice starch, hydroxyethyl starch, Wheat starch, Tapioca starch, or Waxy maize starch.

26. The pharmaceutical dosage form according to claim 22 wherein the nonthermosetting modifier is a maltodextrin.

27. The pharmaceutical dosage form according to claim 21 wherein the non-thermosetting polymer is carboxymethyl cellulose sodium, methyl cellulose, ethylcellulose, hydroxyethylcellulose (HEC), hydroxypropylmethyl cellulose (HPMC), hydroxypropylmethyl cellulose phthalate, cellulose acetate phthalate, noncrystalline cellulose, starch and its derivatives, and sodium starch glycolate or mixtures thereof.

28. The pharmaceutical dosage form according claim 1 which optionally further comprises a sweetener, a disintegrant, a binder, a lubricant, or an opacifier.

29. The pharmaceutical dosage form according to claim 28 wherein the disintegrant is croscarmellose sodium, sodium starch glycolate, sodium carboxymethyl-cellulose, Ac-di-sol®, carboxymethyl-cellulose, veegum, an alginate, agar, guar, tragacanth, locust bean, karaya, pectin, or crospovidone.

30. The pharmaceutical dosage form according to claim 28 wherein the lubricant is glycerol monosterate, stearyl alcohol NF, stearic acid NF, Cab-O-Sil, Syloid, zinc stearate USP, magnesium stearate NF, calcium stearate NF, sodium stearate, cetostrearyl alcohol NF, sodium stearyl fumerate NF, or talc.

31. The pharmaceutical dosage form according to claim 28 wherein the opacifiers is talc USP, calcium carbonate USP, or kaolin USP.

32. The pharmaceutical dosage form according to claim 21 wherein the active pharmaceutical agent is selected from an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antithyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, and a vasodilator.

33. The pharmaceutical dosage form according to claim 21 wherein the solid excipient further comprises a thermoplastic polymer.

34. The pharmaceutical dosage form according to claim 33 wherein the thermoplastic polymer is polyethylene oxide, hydroxypropylcellulose, polyethylene glycol, polyvinyl pyrrolidone, copovidone, or povidone or mixtures thereof.

35. The pharmaceutical dosage form according to claim 34 wherein the polymer is polyethylene oxide, hydroxypropylcellulose, or a mixture thereof.

36. The pharmaceutical dosage form according to claim 21 wherein the non-thermosetting polymerized plastics material contains at least one polyol, and at least one non-thermosetting modifier, and optionally a or a thermosetting polymer.

37. The pharmaceutical dosage form according claim 21 wherein the microcellular polymeric material is a closed cell foam.

38. A pharmaceutical dosage form according to claim 21, in which the homogeneous solid mixture has a sufficiently high solubility in saliva that the dosage form dissolves substantially immediately in the mouth upon oral administration.

39. A pharmaceutical dosage form according to claim 21, in which the voids are in the form of closed cells.

40. A pharmaceutical dosage form according to claim 21, in which the rigid microcellular foam is enclosed within a skin having a density substantially greater than that of the microcellular foam, but having the same composition as that of said solid mixture.

41. A pharmaceutical dosage form according to claim 21, in which the overall density of the dosage form is substantially less than that of stomach fluids, whereby the dosage form is gastro-retentive.

42. A method for making pharmaceutically acceptable dosage forms including a pharmaceutical agent and a non-thermosetting excipient polymer, the method comprising the steps of:
heating the non-thermosetting excipient polymer to a temperature at which the polymer can be molded;
applying pressure to the polymer to maintain the polymer at elevated pressure;
while maintaining the polymer at elevated pressure, forming a single phase solution comprising said polymer and a substance which is substantially non-reactive with said pharmaceutical agent to form a single-phase solution, said substance being a gas under ambient temperature and pressure;
forming the polymer into solid dosage forms by injection molding; and
at a time prior to the forming of the polymer into solid dosage forms, mixing said pharmaceutical agent with the polymer to form a homogeneous mixture;
wherein, in the process of forming the polymer into solid dosage forms, the elevated pressure is reduced to a level at which a very large number of cells is nucleated, each cell containing said gas; and
after the cells are nucleated, the temperature of the polymer is rapidly reduced to limit cell growth.

43. The method according to claim 42, in which the step of mixing said pharmaceutical agent with the polymer to form a homogeneous mixture is carried out prior to the steps of heating and applying pressure.

44. The method according to claim 42, in which said single phase solution is formed by introducing said substance into said polymer by injecting said substance under pressure.

45. The method according to claim 42, in which said substance is introduced into the polymer in the form of a gas.

46. The method according to claim 42, in which said substance is introduced into the polymer in the form of a gas, and the gas introduced into the polymer remains in solution in the polymer while the polymer is under a pressure greater than ambient pressure.

47. The method according to claim 42, in which said substance is introduced into the polymer in the form of a gas, the amount of gas introduced into the polymer is sufficient to form a saturated single phase solution, and the level to which the elevated pressure is reduced is a level at which the single phase solution becomes thermodynamically unstable and gas evolves from the solution in the form of bubbles.

48. The method according to claim 42, in which said substance is introduced into the polymer in the form of a supercritical fluid.

49. The method according to claim 42, in which the pressure and temperature reduction steps are carried out at rates such that the maximum void dimension in the solid dosage form is in the range from about 2 to 100 microns and the void fraction is in the range of about 5 to 95 percent.

50. The method according to claim 42, in which the polymer is formed into pellets by melt extrusion prior to the injection molding step.
